(19) United States
(12) Patent Application Publication
(10) Pub. No.: US 2022/0307094 A1
(43) Pub. Date: Sep. 29, 2022

(54) METHOD FOR DIAGNOSIS OF EARLY AGEING OF THE SKIN

(71) Applicant: L'OREAL, Paris (FR)

(72) Inventors: Philippe BASTIEN, Aulnay-Sous-Bois (FR); Cécile CLAVAUD, Aulnay-Sous-Bois (FR); Nasrine BOUROKBA, Singapore (SG)

(21) Appl. No.: 17/619,154

(22) PCT Filed: Jun. 24, 2020
(86) PCT No.: PCT/EP2020/067699

§ 371 Date: Dec. 14, 2021

(30) Foreign Application Priority Data
Jun. 24, 2019 (FR) FR1906830

(51) Int. Cl. C12Q 1/6895

(52) U.S. Cl. CPC C12Q 1/6895; C12Q 2600/124; C12Q 2600/158

(57) ABSTRACT

The present invention concerns a method for diagnosing early ageing of the skin in a subject, that in particular is pollution-related, comprising a step (a) of determining in a skin sample of the subject the level of at least one marker selected from the group constituted of fungi comprising a nucleic acid encoding an ITS1 («Internal Transcribed Spacer 1») region of sequence being at least 90% identical to sequence SEQ ID NO: 1, SEQ ID NO: 2, SEQ ID NO: 3, SEQ ID NO: 4, SEQ ID NO: 5 or SEQ ID NO: 6, and optionally fungi comprising a nucleic acid encoding an ITS1 region of sequence being at least 90% identical to sequence SEQ ID NO: 7, SEQ ID NO: 8, SEQ ID NO: 9 or SEQ ID NO: 10.

The present invention concerns methods for diagnosis of early ageing of the skin.

Some urban areas are regularly exposed to pollution peaks. Persons in their daily environment. In particular in urban areas, can be subjected to multiple harmful effects on keratin material and in particular on the skin via different airborne pollutants.

Among known pollutants, first mention is made of exhaust gases which have become of major concern in large towns, producing heavy metals but also fine particles containing molecules such as polycyclic aromatic hydrocarbons e.g. benzo-a-pyrene or benzoanthracene.

In particular these pollutants lead at the skin level to the depositing of particles on the epidermal surface and, among other consequences, cause early ageing of the skin marked by the presence of lines and/or wrinkles, large macules, lentigo simplex, red patches and/or complexion of dull and/or heterogeneous appearance.

The present inventors have shown that extended exposure to atmospheric pollution is associated with changes in the fungal microbiome of the skin and with early ageing of the skin.

By identifying exposure to pollution as a factor responsible for early ageing of the skin through changes in the fungal microbiome of the skin, it could be possible to prevent and/or treat this ageing more efficiently by specifically targeting the changes in the fungal microbiome.

There is therefore a major need for methods to diagnose early ageing of the skin that in particular is pollution-related.

The present invention meets this need.

The present invention results from the unexpected finding by the inventors that skin samples from individuals presenting with early ageing of the skin and exposed to chronic pollution (based on the detection of high levels of pollutants in hair samples thereof) have significantly different levels of some fungi compared with individuals not showing said early ageing and not exposed to such pollution.

The present invention therefore concerns a method for diagnosing early ageing of the skin, in particular linked to pollution, in a subject, comprising a step (a) of determining in a skin sample of the subject, in particular of the skin surface of the subject, the level of at least one marker selected from the group constituted of fungi comprising a nucleic acid encoding an ITS1 region («Internal Transcribed Spacer 1») of sequence being at least 90% identical to sequence SEQ ID NO: 1, SEQ ID NO: 2, SEQ ID NO: 3, SEQ ID NO: 4, SEQ ID NO: 5 or SEQ ID NO: 6, and optionally fungi comprising a nucleic acid encoding an ITS1 region of sequence being at least 90% identical to sequence SEQ ID NO: 7, SEQ ID NO: 8, SEQ ID NO: 9 or SEQ ID NO: 10.

In one particular embodiment, said at least one marker is selected from the group constituted of fungi of genus Candida, fungi of the Sclerotiniaceae family, fungi of genus Emericella, fungi of order Hypocreales, fungi of genus Mucor, fungi of genus Sporobolomyces, and optionally fungi of genus Malassezia and fungi of genus Cryptococcus.

In one preferred embodiment, said at least one marker is selected from the group constituted of fungi of genus Candida, fungi of the Sclerotiniaceae family, fungi of genus Emericella, fungi of order Hypocreales, fungi of genus Mucor and fungi of genus Sporobolomyces.

### DETAILED DESCRIPTION OF THE INVENTION

### Marker

The marker used in the context of the invention is selected from the group constituted of fungi comprising a nucleic acid encoding an ITS1 region («Internal Transcribed Spacer 1») of sequence being at least 90% identical to sequence SEQ ID NO: 1, SEQ ID NO: 2, SEQ ID NO: 3, SEQ ID NO: 4, SEQ ID NO: 5 or SEQ ID NO 6, and optionally fungi comprising a nucleic acid encoding an ITS1 region of sequence being at least 90% identical to sequence SEQ ID NO: 7, SEQ ID NO: 8, SEQ ID NO: 9 or SEQ ID NO: 10.

In one particular embodiment, the marker used in the context of the invention is selected from the group constituted of fungi comprising a nucleic acid encoding an ITS1 region of sequence being at least 90% identical to sequence SEQ ID NO: 1, SEQ ID NO: 2, SEQ ID NO: 3, SEQ ID NO: 4, SEQ ID NO: 5 or SEQ ID NO: 6.

By «ITS1 region» or «Internal Transcribed Spacer 1», it is meant herein an internal transcribed spacer (ITS) DNA sequence of the ribosomal RNA gene region positioned between the genes of the small-subunit of ribosomal RNA (rRNA) and of the large-subunit of ribosomal RNA in the chromosome or the corresponding transcribed region in the polycistronic rRNA precursor transcript. More specifically, the ITS1 region is located in eukaryotes between rRNA genes 18S and 5.8S.

Therefore, the marker used in the context of the invention can be selected from the group constituted of fungi comprising an ITS1 region of sequence being at least 90% identical to sequence SEQ ID NO: 1, SEQ ID NO: 2, SEQ ID NO: 3, SEQ ID NO: 4, SEQ ID NO: 5 or SEQ ID NO: 6, and optionally fungi comprising an ITS1 region of sequence being at least 90% identical to sequence SEQ ID NO: 7, SEQ ID NO: 8, SEQ ID NO: 9 or SEQ ID NO: 10. In one particular embodiment, the marker used in the context of the invention is selected from the group constituted of fungi having an ITS1 region of sequence being at least 90% identical to sequence SEQ ID NO: 1, SEQ ID NO: 2, SEQ ID NO: 3, SEQ ID NO: 4, SEQ ID NO: 5 or SEQ ID NO: 6.

In the context of the present invention, «percentage identity» is calculated using global alignment (i.e. the two sequences are compared over their entire sequence). Methods for comparing the identity of two sequences or more are well known to the skilled person. The command «assign_taxonomy.py» in QIIME (version 1.9) against a curated database as described in Findley et al. (2013) Nature 498:367-370 can be used for example.

In one particular embodiment, the marker used in the context of the invention is selected from the group composed of fungi comprising a nucleic acid encoding an ITS1 region, in particular an ITS1 region, of sequence being at least 91% identical, in particular at least 92%, at least 93%, at least 94%, at least 95%, at least 96%, at least 98%, at least 99%, at least 99.5%, at least 99.9% or at least 100% identical to sequence SEQ ID NO: 1, SEQ ID NO: 2. SEQ ID NO: 3, SEQ ID NO: 4, SEQ ID NO: 5 or SEQ ID NO: 6, and optionally fungi comprising a nucleic acid encoding an ITS1 region, in particular an ITS1 region, of sequence being at least 91% identical, in particular at least 92%, at least 93%, at least 94%, at least 95%, at least 96%, at least 98%, at least 99%, at least 99.5%, at least 99.9% or at least 100% sequence Identity with sequence SEQ ID NO: 7, SEQ ID NO: 8, SEQ ID NO: 9 or SEQ ID NO: 10.

In one particular embodiment, the marker used in the context of the invention is selected from the group constituted of fungi comprising a nucleic acid encoding an ITS1 region, in particular an ITS1 region, of sequence being at least 91% Identical, in particular at least 92%, at least 93%, at least 94%, at least 95%, at least 96%, at least 98%, at least 99%, at least 99.5%, at least 99.9% or at least 100% identical to sequence SEQ ID NO: 1, SEQ ID NO: 2, SEQ ID NO: 3, SEQ ID NO: 4, SEQ ID NO: 5 or SEQ ID NO: 6.

Sequence SEQ ID NO: 1 is a sequence representative of the ITS1 of fungi of genus Candida.

Sequence SEQ ID NO: 2 is a sequence representative of the ITS1 of fungi of the Sclerotiniaceae family.

Sequence SEQ ID NO: 3 is a sequence representative of the ITS1 of fungi of genus Emericella.

Sequence SEQ ID NO: 4 is a sequence representative of the ITS1 of fungi of order Hypocreales.

Sequence SEQ ID NO: 5 is a sequence representative of the ITS1 of fungi of genus Mucor.

Sequence SEQ ID NO: 6 is a sequence representative of the ITS1 of fungi of genus Sprobolomyces.

Sequence SEQ ID NO: 7 is a sequence representative the ITS1 of fungi of genus Malassezia (OTU F11231).

Sequence SEQ ID NO: 8 is an alternative sequence representative of the ITS1 of fungi of genus Malassezia (OTU F4665).

Sequence SEQ ID NO: 9 is an alternative sequence representative of the ITS1 of fungi of genus Malassezia (OTU F495).

Sequence SEQ ID NO: 10 is a sequence representative of the ITS1 of fungi of genus Cryptococcus.

Thus, in a particular embodiment, said at least one marker is selected from the group constituted of fungi of genus Candida, fungi of the Sclerotiniaceae family, fungi of genus Emericella, fungi of order Hypocreales, fungi of genus Mucor, fungi of genus Sporobolomyces, and optionally fungi of genus Malassezia and fungi of genus Cryptococcus.

In a preferred embodiment, said at least one marker is selected from the group constituted of fungi of genus Candida, fungi of the Sclerotiniaceae family, fungi of genus Emericella, fungi of order Hypocreales, fungi of genus Mucor and fungi of genus Sporobolomyces.

By «fungi of genus Candida», it is meant herein round or oval-shaped, budding yeasts often accompanied by mycelian or pseudo-mycelian filaments, of which the representative species is Candida albicans, forming a commensal in healthy persons in the mouth, on the skin, in the digestive system and in vaginal flora, depending on species.

By «fungi of the Sclerotiniaceae family», it is meant herein fungi belonging to the order of Helotiales which propagate by sclerotia or stromata.

By «fungi of genus Emericella», it is meant herein teleomorphs of fungi of genus Aspergillus.

By «fungi of order Hypocreales», it is meant herein fungi of the Hypocreomycetidae sub-class, the fruiting bodies of which, when the teleomorph is known, are perithecia.

By «fungi of genus Mucor», it is meant herein fungi of order Mucorales, typically forming white-to-beige or grey colonies, of rapid growth and having pores that can be simple or branched and form an apical, globular sporangium.

By «fungi of genus Sporobolomyces», it is meant herein anamorphic yeasts of order Sporidibolales of which the teleomorph forms are included in genus Sporidibolus.

By «fungi of genus Malassezia», it is meant herein yeasts of the Malasseziaceae family naturally found on the skin surface in numerous animals including humans.

By «fungi of genus Cryptococcus», it is meant herein yeasts of the Tremellaceae family, the sexual or teleomorph forms of which are of genus Filobasidiella.

### Diagnosis Method

The diagnosis method of the invention is a method for diagnosis of early ageing of the skin that is in particular pollution-related.

By «early ageing of the skin», it is particularly meant the first signs of skin ageing which generally affect persons in the 25-45 age range, and notably translates as the onset of lines and complexion of dull and/or heterogeneous appearance.

Early ageing of the skin is particularly evidenced by the presence of lines and/or wrinkles, large macules, lentigo simplex, red patches and/or complexion of dull and/or heterogeneous appearance.

Therefore, in a particular embodiment, early ageing of the skin includes the presence of lines and/or wrinkles, large macules, lentigo simplex, red patches and/or complexion of dull and/or heterogeneous appearance.

The skin is more particularly facial skin, especially the skin of cheeks and/or forehead, the skin of neckline, the skin of neck, the skin of arms and forearms. More preferably, the skin is facial skin and in particular cheek and/or forehead skin.

In a particular embodiment, early ageing of the skin is pollution-related, in particular due to pollution.

By «pollution», it is meant herein chronic exposure to particulate matter, in particular to polycyclic aromatic hydrocarbons (PAHs).

In a particular embodiment, pollution is exposure to particulate matter, in particular to PAHs leading to the following level of PAHs and PAH metabolites in the hair of the subject:
level of 2-OH-phenanthrene at least 1.8 times higher than a control level;
level 3-OH-fluorene at least 1.6 times higher than a control level;
level of 3-OH-phenanthrene at least 1.7 times higher than a control level;
level of B-b-fluoranthene at least 1.8 times higher than a control level;
level of benzo-g-h-i-perylene at least 1.7 times higher than a control level;
level of fluoranthene at least 2 times higher than a control level; and/or
level of pyrene at least 1.6 times higher than a control level,

the control level typically being the level of said compound in the hair of a subject living in a city with low pollution, in particular a city having an air quality index below 100 for less than 100 days, in particular less than 85 days over a one-year period.

The diagnosis method of the Invention comprises a step (a) of determining, in a skin sample of the subject, in particular a surface skin sample, the level of at least one marker selected from the group constituted of fungi as defined in the above «Marker» section.

In a particular embodiment, the level of said at least one marker in the sample is the relative abundance of said at least one marker in the sample.

By «relative abundance», it is meant herein the relative amount in percentage of a given taxon relative to the total number of taxa in the sample.

The level of said at least one marker can be determined using any suitable technique.

In a particular embodiment, the level of said at least one marker, in particular the relative abundance of said at least one marker, is determined by measuring the level of the corresponding ITS1 DNA region.

Preferably the level of said at least one marker is determined by PCR amplification combined with sequencing, particularly high-throughput sequencing, of the ITS1 DNA region.

Typically, the fungal genomic DNA present in the skin sample is extracted and then subjected to PCR using primers targeting the fungal ITS1 DNA region, in particular as described in Leung et al. (2016) Microblome 4:46. The ITS1 DNA amplicons obtained were subjected to sequencing allowing identification of the corresponding fungi and measurement of the relative abundance of each identified fungal ITS1DNA.

In a particular embodiment, the diagnosis method of the invention further comprises the steps consisting in:
(b) comparing the level of said at least one marker measured at step (a) with a control; and
(c) on the basis of the comparison at step (b), determining whether the skin of the subject shows early ageing, in particular pollution-related.

In a particular embodiment, the control is a reference value.

In a particular embodiment, the reference value is determined by the mean value of the level of said marker in a determined population, for example a population in a determined age group and/or having a defined skin type.

In a particular embodiment, the reference value is the mean value of the level of said marker in a population of subjects, in particular subjects as defined below, living in a city with low pollution, in particular a city having an air quality index below 100 for less than 100 days, in particular less than 85 days over a one-year period.

In a particular embodiment, the subject's skin is diagnosed as showing early ageing, in particular pollution-related, when:
the level of fungi of genus Candida, in particular the relative abundance thereof, determined in the skin sample of the subject is higher, in particular significantly higher than a control level;
the level of fungi of the Sclerotiniaceae family, in particular the relative abundance thereof, determined in the skin sample of the subject is higher, in particular significantly higher than a control level;
the level of fungi of genus Emericella, in particular the relative abundance thereof, determined in the skin sample of the subject is higher, in particular significantly higher than a control level;
the level of fungi of order Hypocreales, in particular the relative abundance thereof, determined in the skin sample of the subject is higher, in particular significantly higher than a control level;
the level of fungi of genus Mucor, in particular the relative abundance thereof, determined in the skin sample of the subject is higher, in particular significantly higher than a control level;
the level of fungi of genus Sporobolomyces, in particular the relative abundance thereof, determined in the skin sample of the subject is higher, in particular significantly higher than a control level;
the level of fungi of genus Malassezia, in particular the relative abundance thereof, determined in the skin sample of the subject is lower, in particular significantly lower than a control level; and/or
the level of fungi of genus Cryptococcus, in particular the relative abundance thereof, determined in the skin sample of the subject is higher, in particular significantly higher than a control level,

the control level typically being the level of said marker, in particular the relative abundance of said marker, in a skin sample of a subject living in a city with low pollution, in particular a city having an air index quality below 100 for less than 100 days, in particular less than 85 days over a one-year period; and

the marker levels typically being determined by measuring the level of the corresponding ITS1 DNA region, in particular by PCR amplification combined with sequencing of the ITS1 DNA region, typically as described above.

By «significantly lower» in the meaning of the invention it is meant a statistically significant reduction in the level of the marker compared with the control level.

By «significantly higher» in the meaning of the invention it is meant a statistically significant increase in the level of the marker compared with the control level.

In a preferred embodiment, the skin sample of the subject used in the diagnosis method of the invention is a sample taken, preferably non-invasively, from the skin of the subject, preferably the subject's face, in particular on the cheek and/or forehead of the subject. Preferably, the skin sample is from the stratum corneum, in particular from the surface of the stratum corneum.

The stratum corneum is the layer the furthest distant from the epidermis and comprises the skin surface. It is mainly composed of dead cells.

In an embodiment, the diagnosis method of the invention comprises a step of taking a skin sample from the subject in particular from the surface of the subject's skin. This step is preferably performed non-invasively, and in particular does not require a local anaesthetic.

In a preferred embodiment, the sample-taking step is performed by rubbing the surface of the skin in particular with a moist cotton swab.

In a particular embodiment, the skin sample is thus taken by rubbing the surface of the skin.

By «subject», it is meant herein a human being, preferably aged 25 to 45 years. Preferably the subject is female. Preferably the subject is of Asian type.

In a particular embodiment, the age of the subject is between 25 and 45 years.

The present invention also concerns a method of cosmetic treatment of skin showing early ageing, in particular pollution-related, in a subject, said method comprising the following steps:
A) diagnosing the subject as showing early ageing of the skin, in particular pollution-related, by implementing the diagnosis method of the invention;
B) if the subject is diagnosed as showing early ageing of the sin, in particular pollution-related, treating the skin of said subject with a cosmetic composition allowing reducing and/or slowing early ageing of the skin.

In a particular embodiment, the cosmetic composition used in the treatment method of the invention comprises probiotics and/or prebiotics, in particular enabling promoting the presence of commensal flora.

The present invention is described in more details in the FIGURE and examples given below.

### Description of Sequences

| OTU | Taxonomy | SEQ ID | Sequence |
|---|---|---|---|
| F14 | Candida | 1 | GTAAAAGTCGTAACAAGGTTTCCGTAGGTGAACCTGCGGAA |
| | | | GGATCATTACAGAATGAAAAGTGCTTAACTGCATTTTTTCTT |
| | | | ACACATGTGTTTTTCTTTTTTTGAAAACTTTGCTTTGGTAGG |
| | | | CCTTCTATATGGGGCCTGCCAGAGATTAAACTCAACCAAAT |
| | | | TTTATTTAATGTCAACCGATTATTTAATAGTCAAAACTTTCAA |
| | | | CAACGGATCTCTTGGTTCTCGCATCGATGAAGAACGCAGCG |
| F67 | Sclerotiniaceae | 2 | GTAAAAGTCGTAACAAGGTTTCCGTAGGTGAACCTGCGGAA |
| | | | GGATCATTACAGAGTTCATGCCCGAAAGGGTAGACCTCCCA |
| | | | CCCTTGTGTATTATTACTTTGTTGCTTTGGCGAGCTGCCTTC |
| | | | GGGCCTTGTATGCTCGCCAGAGAATACCAAAACTCTTTTTA |
| | | | TTAATGTCGTCTGAGTACTATATAATAGTTAAAACTTTCAACA |
| | | | ACGGATCTCTTGGTTCTGGCATCGATGAAGAACGCAGCGAA |
| | | | A |
| F26 | Emericella | 3 | GTAAAAGTCGTAACAAGGTTTCCGTAGGTGAACCTGCGGAA |
| | | | GGATCATTGTCGTGACCCTGACCAAAACAGACCGCGCACG |
| | | | CGTCATCCAATCCGTCGGCGACGGCACCGTCCGTCGCTCG |
| | | | GCCAATGCCTCGACCACCTCCCCTCCTCGGAGCGGGTGGG |
| | | | GGCTCGGGGTAAAAGAACCCACGGCGCCGAAGGCGTCAA |
| | | | GGAACACTGTGCCTAACCCGGGGGCATGTCTAGCTTGCTA |
| | | | GCCGTCCCTC |
| F37 | Hypocreales | 4 | GTAAAAGTCGTAACAAGGTTTCCGTTGGTGAACCAGCGGAG |
| | | | GGATCATTATAGAGTGTAAACTCCCTAACCTTTGTGAACATA |
| | | | CCTGTTGCTTCGGCGGTCCTCACCGGCCGCCGAAGGCCTA |
| | | | TATTCTTGAATTTACATGAATTTCTGAGTATCAAACAAAAATA |
| | | | AATAAAAACTTTCAGCAACGGATCTCTTGGCTCTGGCATCG |
| | | | ATGAAGAACGCAGCGAAATGCGATAAGTAATGTGAATTGCA |
| | | | GA |
| F89 | Mucor | 5 | GTAAAAGTCGTAACAAGGTTTCCGTAGGTGAACCTGCGGAA |
| | | | GGATCATTAAATAATCAATAATTTTGGCTTGTCCATCATTATC |
| | | | TATTTACTGTGAAACGTATTATTACTTGACGCCTGAGGGATG |
| | | | TTCCACTGCTATAAGGATAGGCAGCGGAAATGTTAACCGAG |
| | | | TCATAATCAAGCTTAGGCTTGGTATCCTATTATTATTTACCA |
| | | | AAAGAATTCAGAATTAATATTGTAACATAGACGTAAAAAAT |
| F70 | Sporobolomyces | 6 | GTAAAAGTCGTAACAAGGTTTCCGTAGGTGAACCTGCGGAA |
| | | | GGATCATTAGTGAATAAATAGGGTGTCCAATTTAACTTGGAA |
| | | | CCCGACCTTCTCACATCTAACCCTGTGCATCTGTATATAATG |
| | | | GCGAGCAATTTTCGAATTGTGAGCCATTTCACTTTATAAACA |
| | | | CTAGTCTATGAATGTAAAATTTTTATAACAATAAAAACTTTCA |
| | | | ACAACGGATCTCTTGGCTCTCGCATCGATGAAGAACGCAG |
| F11231 | Malassezia | 7 | GTAAAAGTCGTAACAAGGTTTCTGTAGGTGAATCTGCAGGA |
| | | | GGATCATCAGTGATGATTTGGGCAGGCCATACGGACGCCA |
| | | | AAAAGTGTCCCTGGCCGCCTACACCCACTATACATCCACAA |
| | | | ACCCGTGTGCACTGCCTIGGAGAAAGGCTTCAGAGAAGTTT |
| | | | TTGTGGCCTCTCTTGGGGTCTTTCTTCGCTACAGACTCGAA |
| | | | TGGTTAGTATGAACGTGGAACTTGGTTGGACCGTCACTGGC |
| | | | CAAAA |
| F4665 | Malassezia | 8 | GTAMGTCGTAACAAGGTTTCTGTAGGTGAACCTGCAGAAG |
| | | | GATCATTAGTGAAGATTTGGGCTGGCCATACGGACGCCAAA |
| | | | AAGTGTCCCTGGCCGCCTCCGCCCACTATACCTCCACAAAC |
| | | | CCGTGTTCACTGTCTIGGAGAAAGGCTTCAGAGAAGTTTTT |
| | | | TGTGGCCTCTCTTGGGGTCTTTCTTCGCTACAAAATCGAAT |
| | | | GGTTAGTATGAACGTGGAACTTGGTTGGACCGTOACTGGC |
| | | | CAACA |
| F495 | Malassezia | 9 | GTAAAAGICGTAACAAGGITTCTGTAGGTGAACCTGCAGAA |
| | | | GGATCATTAGTGAAGATTIGGGCAGGCCATACGGACGCCA |
| | | | CAAAGTGTCCCTGGCCGCCTACACCCACTATACATCCACAA |
| | | | ACCCGTGTGCACTGTOTTGGAGWGGCTTCTTGAGAAGTT |
| | | | TTGTGGCCTCTCTTGGGGTCTTTCTTCTTCGCTACAAACTC |
| | | | GAATGGTTAGTATGAACGTGGAACTTGGTTGGACCGTCACT |
| | | | GGCCA |
| F28 | Cryptococcus | 10 | GTAAAAGTCGTAACAAGGTTTCCGTAGGTGAACCTGCGGAA |
| | | | GGATCATTAATGAATTTAGATTGAACCATAGGCGAAAGCCA |
| | | | GIGGTICTTCTTTCATATCCATAACACCTGTGOACTGITGGA |
| | | | TGCTTGCATCCACTTTTAAACTAAACATTATTGTAACAAATGT |
| | | | AGTOTTATTATAACATAATAWCTITCAACAACGGATCTCTT |
| | | | GGCTCTCGCATCGATGAAGAACGCAGCGAAATGCGATAAG |

### BRIEF DESCRIPTION OF THE FIGURES

FIG. 1 shows clustering of fungi and early ageing. The grey circles correspond to individuals having wrinkles and hyperpigmented spots. The left-hand ellipse corresponds to the cluster: «Early ageing»: n (total)=90 (53 Baoding/37 Dalian) 59% vs 41%.

### EXAMPLE

The example below shows the identification of a signature comprising 8 fungi which are significantly modulated in skin samples of individuals exposed to chronic pollution (on the basis of detection of high levels of pollutants in hair samples of the individuals).

### Materials and Methods

### Main Steps:

skin sampling
profiling fungal ITS1 rDNA
determining the relative abundance of microbiome markers
statistical study
diagnosis of early ageing of the skin.

### Description of Subjects

All the subjects in each city came to the facilities in Baoding and Dalian (China). Skin samples were collected in 204 Chinese women in good health, being 25 to 45 years-old, among whom 102 lived in the relatively rural and industrial city of Baoding, a city in the north of China in the Hebei province recording high levels of air pollution (about 90 μg PM_{2.5}/m³ air), and 102 living in Dalian, a city in the north of China, urbanised and modern in the province of Liaoning with a lower degree of recorded air pollution (about 30 μg PM_{2.5}/m³ air). These cities are located at the same latitude and have shared a similar climate and equivalent UV exposure (UV index) over the last 15 years.

The participants living in the two cities were assessed for their exposure to PAHs (polycyclic aromatic hydrocarbons) in 12 cm hair samples (reflecting the extent of exposure over a one-year period) (Palazzi et al. (2018) Env. Int. 121:1341-1354). Specifically, in Baoding, the median concentration was 1.5 to 2.8 times higher for parent PAHs and 1.1 to 2.3 times higher for PAH metabolites than the concentration in Dalian. Among quantified parent PAHs, higher levels were observed for phenanthrene, fluoranthene, pyrene, fluorene, acenaphthylene and anthracene, whereas for the PAH metabolites the levels of 9-OH fluorene, 2-OH-naphthalene and 1-OH-anthracene were higher (Palazzi et al. (2018) Env. Int. 121:1341-1354).

On clinical level, increased severity was observed for almost all facial signs including wrinkles and pigmentary disorders in individuals living in Baoding. In addition, discriminant analysis of the subjects was conducted using dermatological evaluation data (Bourokba et al., poster presented at the 76^{th} annual conference of the Society for Investigative Dermatology, Portland, Ore. 26-29 Apr. 2017). This analysis led to the definition of an «early ageing» cluster, corresponding to n=90 women out of 204 (53 from Baoding and 37 from Dalian). The average age of these women was 36 years and they showed increased levels of wrinkles and pigmentary disorders (large macules, lentigo simplex, red patches).

### Subjects and Sample Collection

None of the participants received systemic antibiotics or antifungals for one month before sampling, none had a severe skin disorder or had used skin or systemic depigmenting/whitening treatments for three months before sampling, or an exfoliative product one month before sampling. They were requested to use a provided neutral soap not containing any antibacterial compounds for face washing for 3 days (once a day) before sampling. The last shampoo and last soap were applied respectively 48 and 24 h before sampling. No other product was authorised on the scalp, hair and face until samples had been taken.

Microbiota sampling was performed in a room with controlled atmosphere at 22° C. and 60% humidity. The samples for microbiome analysis were collected using dry, sterile cotton buds which were heated to 150° C. and pre-moistened with ST solution (0.15 M NaCl with 0.1% Tween 20). For cheek samples, the swabs were immersed in collection buffer and firmly rubbed on the cheek for 60 seconds to cover a surface of 1 cm×2 cm. After sampling, each cotton bud was placed in a microtube, immediately frozen in liquid nitrogen, and stored at −80° C. before extracting genomic DNA (gDNA).

Profiling of Fungal ITS1 rDNA
Preparation of an amplicon sample for ITS sequencing.

The gDNA was extracted using the PowerSoil DNA® isolating kit (MO BIO Laboratories, Carlsbad, Calif., USA) following the manufacturer's instructions with the modifications described in Leung et al. (2014) Appl. Environ. Microbiol. 80:6760-6770. In addition, after C6 elution, the eluate was passed an additional time through the same column filter to increase yield. Negative water controls without DNA were extracted in parallel. Each gDNA sample was subjected to PCR in triplicate with primers targeting the ITS1 region as described in Leung et al. (2016) Microbiome 4:46. For analysis of the ITS1 region, the amplicon PCR and indexing PCR were prepared on a PCR 7500 Fast Real-Time PCR System (Applied Biosystems, Foster City, Calif., USA), and the amplicons were purified with DNA/RNA purification beads (SeqMatic, Fremont, Calif., USA). Preparation of the library and paired-end sequencing of the fungal nucleic acids of 250 bp on Illumina Miseq® platform were performed by SeqMatic LLC (Fremont, Calif., USA).
Processing of the ITS sequence and bioinformatic analysis

Fungal reads respectively paired in.fastq format were merged using the command «-fastq_mergeairs» in USEARCH. The merged reads were filtered for quality control using the command «-fastq_filter» in USEARCH, with a maximum expected error rate of 0.01.

The merged reads were cut-off at 450 bp and the shortest reads were discarded. The filtered reads were subjected to OTU grouping with 97% sequence identity using the UPARSE algorithm (Edgar (2013) Nature Methods 10:996-998). The fungal OTUs were interrogated against an organized fungal database designed for monitoring the skin microbiome (Findley et al. (2013) Nature 498:367-370). Detection of chimera was performed using UCHIME2 (Edgar (2016) bioRxiv 074252) under high confidence mode. The OTUs in the taxonomic lines present in more than 5% of negative controls were considered to be potential contaminants (Leung et al. (2018) Microbiome 6:26), and were removed from the dataset. In addition, chimeric OTUs, OTUs of chloroplasts and of mitochondria were also removed. After quality control and removal of undesirable reads, a total of 14649172 fungal reads was retained.

### Statistical Analysis

Two statistical approaches were used: a hypothesis test on OTU abundance and multivariate analysis using hierarchical multiblock analysis (MAXVAR-A).

The result of these tests is qualitative: normal skin or skin showing early ageing.
Method 1: comparison of OTU abundance per marker or in combination.

With regard to the processing of relative abundance data, pre-filtering was performed on OTU relative abundance. OTUs with relative abundance lower than 0.1% among all the individuals were discarded. Also, CSS («Cumulative sum scaling») standardisation was applied which corrects biases in the assessment of differential abundance induced by TSS («Total sum scaling») standardisation. CSS standardisation of data was applied using the R package metagenomeSeq (http://www.cbcb.umd.edu/software/metagenomeSeq).

The mean difference between the two groups in terms of OTU abundance was evaluated by statistical test procedure using the v-test. V-test values correspond to the comparison between the mean OTU abundance in the group of interest (i.e. early ageing) (column: Mean.in.category) and the mean OTU abundance of the total population (column: Overall.mean). A positive value Indicates that OTU abundance is higher in the group of interest and a negative value indicates lower abundance. A p-value associated with each v-test value was also calculated (column: p.value).

The mean OTU abundance in subjects other than in the «early ageing» group was calculated (column: mean.in.cluster1.3.4). OTU abundance, after CSS standardisation, per group, and the threshold values are given in the table below.

| | | | | R1 = | |
|---|---|---|---|---|---|
| OTUs | Genus | N | v.test | Mean.in.category | Overall.mean |
| F11231 | Malassezia | 90 | −3.217047857 | 5.344342385 | 5.843403089 |
| F14 | Candida | 90 | 2.313612741 | 8.221231167 | 7.507521539 |
| F26 | Emericella | 90 | 2.079601491 | 4.908542505 | 4.358347787 |
| F28 | Cryptococcus | 90 | 2.432235172 | 6.430968209 | 5.768489628 |
| F37 | unclassified_Hypocreales_genus | 90 | 2.011446181 | 4.145301468 | 3.542348539 |
| F4665 | Malassezia | 90 | −2.046548822 | 6.894411374 | 7.089832626 |
| F495 | Malassezia | 90 | 2.492228817 | 6.592514446 | 5.947327592 |
| F67 | unclassified_Sclerotiniaceae_genus | 90 | 2.252923104 | 4.297122808 | 3.658265936 |
| F70 | Sporobolomyces | 90 | 1.967496681 | 2.894134872 | 2.405374295 |
| F89 | Mucor | 90 | 1.992780973 | 1.767717962 | 1.378751049 |

| | | | | | OTU |
|---|---|---|---|---|---|
| | | | | | threshold |
| | R2 = | | | | value for |
| OTUs | mean.in.cluster1.3.4 | sd.in.category | Overall.sd | p.value | early ageing |
| F11231 | 6.240885065 | 1.843859765 | 1.967675918 | 0.001295170 | <R2 |
| F14 | 6.939080243 | 3.699983995 | 3.912808610 | 0.020688974 | >R2 |
| F26 | 3.920139604 | 3.251421014 | 3.355784120 | 0.037562100 | >R2 |
| F28 | 5.240851820 | 3.052527138 | 3.454808623 | 0.015005961 | >R2 |
| F37 | 3.062120543 | 3.947335323 | 3.802180320 | 0.044278350 | >R2 |
| F4665 | 7.245477872 | 1.204036406 | 1.211176404 | 0.040702404 | <R2 |
| F495 | 5.433461956 | 3.129962031 | 3.283638313 | 0.012694423 | >R2 |
| F67 | 3.149441879 | 3.484592943 | 3.596788655 | 0.024263998 | >R2 |
| F70 | 2.016095960 | 3.437818152 | 3.150938145 | 0.049125978 | >R2 |
| F89 | 1.068954393 | 2.601905214 | 2.475772927 | 0.046285436 | >R2 |

Method 2: Comparison of fungal relative abundance profile in relation to historical grouping of subjects versus PAH compounds (Baoding/Dalian database).

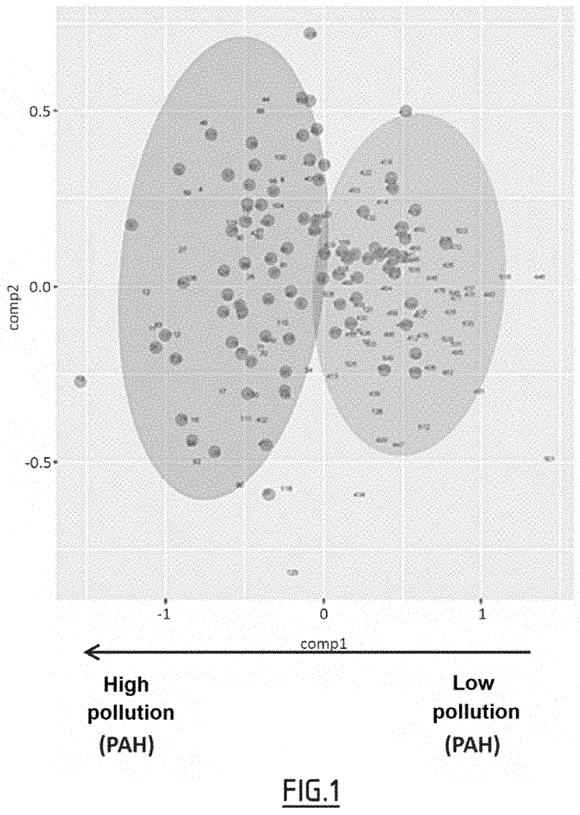

With regard to analysis of global correlation with PAH, pre-filtering was performed on OTU relative abundance. OTUs with relative abundance lower than 0.1% among all individuals were removed. Also, CSS standardisation («Cumulative sum scaling u) was applied which corrects biases in the assessment of differential abundance induced by TSS («Total sum scaling») standardisation. Among the OTUs, 69 were selected for the fungi. PAH measurements were log-transformed to follow Gaussian distribution. A total of 202 individuals with OTU and PAH data were included in the analysis. With a view to variable selection, Sparse Canonical Correlation Analysis (sCCA) was performed to select the OTU and PAH descriptors which were active in relationships between blocks. The sparsity parameters of sCCA were selected via permutation procedure using the MutiCCA.permute function of the PMD R package (Witten et al. (2009) Biostatistics 10:515-534; Tenenhaus et al. (2014) Biostatistics 15:569-583). Finally, to obtain common representation of individuals in the 2 blocks, hierarchical multi-block analysis (MAXVAR-A) was performed using the RGCCA R package (Tenenhaus et al. (2017) Psychometrika 82:737-777). Subjects showing signs of early ageing are surrounded by a circle in FIG. 1.

For diagnosis, the new profile must be compared with the grouping. If it is contained within the left-hand ellipse, the subject tests positive and shows early ageing of the skin.

1. A method for diagnosis of early ageing of the skin in a subject, comprising a step (a) of determining in a skin sample of the subject the level of at least one marker selected from the group constituted of fungi comprising a nucleic acid encoding an ITS1 («Internal Transcribed Spacer 1») region of sequence at least 90% identical to sequence SEQ ID NO: 1, SEQ ID NO: 2, SEQ ID NO: 3, SEQ ID NO: 4, SEQ ID NO: 5 or SEQ ID NO: 6, and optionally fungi comprising a nucleic acid encoding an ITS1 region of sequence at least 90% identical to sequence SEQ ID NO: 7, SEQ ID NO: 8, SEQ ID NO: 9 or SEQ ID NO: 10.

2. The method according to claim 1, wherein said at least one marker is selected from the group constituted of fungi of genus Candida, fungi of the Sclerotiniaceae family, fungi of genus Emericella, fungi of order Hypocreales, fungi of genus Mucor, fungi of genus Sporobolomyces, and optionally fungi of genus Malassezia and fungi of genus Cryptococcus.

3. The method according to claim 1, wherein said at least one marker is selected from the group constituted of fungi of genus Candida, fungi of the Sclerotiniaceae family, fungi of genus Emericella, fungi of order Hypocreales, fungi of genus Mucor and fungi of genus Sporobolomyces.

4. The method according to claim 1, the method further comprising the steps consisting in:
(b) comparing the level of said at least one marker measured at step (a) with a control; and
(c) on the basis of the comparison at step (b), determining whether the skin of the subject shows early ageing.

5. The method according to claim 1, wherein the level of said at least one marker is determined by measuring the level of the corresponding ITS1 DNA region.

6. The method according to claim 5, wherein the level of said at least one marker is determined by PCR amplification combined with sequencing of the ITS1 DNA region.

7. The method according to claim 1, wherein the skin sample is taken by rubbing the surface of the skin.

8. The method according to claim 1, wherein early ageing of the skin is pollution-related.

9. The method according to claim 1, wherein the subject is aged between 25 and 45 years.

10. The method according to claim 1, wherein early ageing includes the presence of lines and/or wrinkles, large macules, lentigo simplex, red patches and/or a complexion that is dull and/or heterogeneous.

11. The method according to claim 2, the method further comprising the steps consisting in:
(b) comparing the level of said at least one marker measured at step (a) with a control; and
(c) on the basis of the comparison at step (b), determining whether the skin of the subject shows early ageing.

12. The method according to claim 3, the method further comprising the steps consisting in:
(b) comparing the level of said at least one marker measured at step (a) with a control; and
(c) on the basis of the comparison at step (b), determining whether the skin of the subject shows early ageing.

13. The method according to claim 2, wherein the level of said at least one marker is determined by measuring the level of the corresponding ITS1 DNA region.

14. The method according to claim 3, wherein the level of said at least one marker is determined by measuring the level of the corresponding ITS1 DNA region.

15. The method according to claim 4, wherein the level of said at least one marker is determined by measuring the level of the corresponding ITS1 DNA region.

16. The method according to claim 2, wherein the skin sample is taken by rubbing the surface of the skin.

17. The method according to claim 3, wherein the skin sample is taken by rubbing the surface of the skin.

18. The method according to claim 4, wherein the skin sample is taken by rubbing the surface of the skin.

19. The method according to claim 5, wherein the skin sample is taken by rubbing the surface of the skin.

20. The method according to claim 6, wherein the skin sample is taken by rubbing the surface of the skin.


SEQUENCE LISTING (10 sequences)

SEQ ID NO: 1 - DNA, 250 bp, Artificial Sequence
  ITS1 sequence of genus Candida
gtaaaagtcgtaacaaggtttccgtaggtgaacctgcggaaggatcattacagaatgaaaagtgcttaactgcattttttcttacacatgtgtttttctttttttgaaaactttgctttggtaggccttctatatggggcctgccagagattaaactcaaccaaattttatttaatgtcaaccgattatttaatagtcaaaactttcaacaacggatctcttggttctcgcatcgatgaagaacgcagcg

SEQ ID NO: 2 - DNA, 250 bp, Artificial Sequence
  ITS1 sequence of the family Sclerotiniaceae
gtaaaagtcgtaacaaggtttccgtaggtgaacctgcggaaggatcattacagagttcatgcccgaaagggtagacctcccacccttgtgtattattactttgttgctttggcgagctgccttcgggccttgtatgctcgccagagaataccaaaactctttttattaatgtcgtctgagtactatataatagttaaaactttcaacaacggatctcttggttctggcatcgatgaagaacgcagcgaaa

SEQ ID NO: 3 - DNA, 250 bp, Artificial Sequence
  ITS1 sequence of genus Emericella
gtaaaagtcgtaacaaggtttccgtaggtgaacctgcggaaggatcattgtcgtgaccctgaccaaaacagaccgcgcacgcgtcatccaatccgtcggcgacggcaccgtccgtcgctcggccaatgcctcgaccacctcccctcctcggagcgggtgggggctcggggtaaaagaacccacggcgccgaaggcgtcaaggaacactgtgcctaacccgggggcatgtctagcttgctagccgtccctc

SEQ ID NO: 4 - DNA, 250 bp, Artificial Sequence
  ITS1 sequence of order Hypocreales
gtaaaagtcgtaacaaggtttccgttggtgaaccagcggagggatcattatagagtgtaaactccctaacctttgtgaacatacctgttgcttcggcggtcctcaccggccgccgaaggcctatattcttgaatttacatgaatttctgagtatcaaacaaaaataaataaaaactttcagcaacggatctcttggctctggcatcgatgaagaacgcagcgaaatgcgataagtaatgtgaattgcaga

SEQ ID NO: 5 - DNA, 250 bp, Artificial Sequence
  ITS1 sequence of genus Mucor
gtaaaagtcgtaacaaggtttccgtaggtgaacctgcggaaggatcattaaataatcaataattttggcttgtccatcattatctatttactgtgaaacgtattattacttgacgcctgagggatgttccactgctataaggataggcagcggaaatgttaaccgagtcataatcaagcttaggcttggtatcctattattatttaccaaaagaattcagaattaatattgtaacatagacgtaaaaaat

SEQ ID NO: 6 - DNA, 250 bp, Artificial Sequence
  ITS1 sequence of genus Sporobolomyces
gtaaaagtcgtaacaaggtttccgtaggtgaacctgcggaaggatcattagtgaataaatagggtgtccaatttaacttggaacccgaccttctcacatctaaccctgtgcatctgtatataatggcgagcaattttcgaattgtgagccatttcactttataaacactagtctatgaatgtaaaatttttataacaataaaaactttcaacaacggatctcttggctctcgcatcgatgaagaacgcag

SEQ ID NO: 7 - DNA, 250 bp, Artificial Sequence
  ITS1 sequence of genus Malassezia (OTU F11231)
gtaaaagtcgtaacaaggtttctgtaggtgaatctgcaggaggatcatcagtgatgatttgggcaggccatacggacgccaaaaagtgtccctggccgcctacacccactatacatccacaaacccgtgtgcactgccttggagaaaggcttcagagaagtttttgtggcctctcttggggtctttcttcgctacagactcgaatggttagtatgaacgtggaacttggttggaccgtcactggccaaaa

SEQ ID NO: 8 - DNA, 250 bp, Artificial Sequence
  Alternative ITS1 sequence of genus Malassezia (OTU F4665)
gtaaagtcgtaacaaggtttctgtaggtgaacctgcagaaggatcattagtgaagatttgggctggccatacggacgccaaaaagtgtccctggccgcctccgcccactatacctccacaaacccgtgttcactgtcttggagaaaggcttcagagaagttttttgtggcctctcttggggtctttcttcgctacaaaatcgaatggttagtatgaacgtggaacttggttggaccgtcactggccaaca

SEQ ID NO: 9 - DNA, 250 bp, Artificial Sequence
  Alternative ITS1 sequence of genus Malassezia (OTU F495)
gtaaaagtcgtaacaaggtttctgtaggtgaacctgcagaaggatcattagtgaagatttgggcaggccatacggacgccacaaagtgtccctggccgcctacacccactatacatccacaaacccgtgtgcactgtcttggagaaaggcttcttgagaagttttgtggcctctcttggggtctttcttcttcgctacaaactcgaatggttagtatgaacgtggaacttggttggaccgtcactggcca

SEQ ID NO: 10 - DNA, 250 bp, Artificial Sequence
  ITS1 sequence of genus Cryptococcus
gtaaaagtcgtaacaaggtttccgtaggtgaacctgcggaaggatcattaatgaatttagattgaaccataggcgaaagccagtggttcttctttcatatccataacacctgtgcactgttggatgcttgcatccacttttaaactaaacattattgtaacaaatgtagtcttattataacataataaaactttcaacaacggatctcttggctctcgcatcgatgaagaacgcagcgaaatgcgataag